(19) United States
(12) Patent Application Publication
(10) Pub. No.: US 2012/0225455 A1
(43) Pub. Date: Sep. 6, 2012

(54) Method for Producing Sugar

(75) Inventor: Seiji Fukuhara, Ibaraki (JP)

(73) Assignee: Asahi Groups Holdings, Ltd., Tokyo (JP)

(21) Appl. No.: 13/509,553

(22) PCT Filed: Nov. 18, 2010
(86) PCT No.: PCT/JP2010/070530

§ 371 Date: May 11, 2012

(30) Foreign Application Priority Data
Nov. 26, 2009 (JP) 2009-268829

(51) Int. Cl. C07H 1/08; C07H 3/00; C12P 19/00

(52) U.S. Cl. 435/72; 536/128

(57) ABSTRACT

Disclosed is a process for producing a sugar, whereby any non-sugar component can be removed from a sugar solution with high efficiency and the color value of the sugar solution can be reduced. Specifically disclosed is a process for producing sugar, which is characterized by comprising: adding ethanol to a sugar solution produced by squeezing a plant; removing precipitates produced by the addition of ethanol from the solution; and crystallizing sugar from the sugar solution from which the precipitates have been removed.

### TECHNICAL FIELD

The present invention relates to a method for producing sugar, and more specifically to a method for efficiently producing sugar and ethanol.

### BACKGROUND ART

When sugar or ethanol is produced by use of sugarcane as a raw material, a process of clarifying a squeezed juice is important. If soluble solid components (proteins, amino acids, minerals, and the like) and suspended substances in the squeezed juice can be sufficiently removed in the clarification process, the crystal yield of sugar can be improved. In addition, the productivity of ethanol can also be improved, because the salt concentration in molasses is lowered. In a conventional process of clarifying sugarcane, soluble solid components are precipitated by a heat treatment and addition of a chemical liquid of lime or the like, and then non-sugar components are separated and removed together with suspended substances, in general (see, for example, Chen, J. C. P. and Chou, C. C. (1993), Cane Sugar Handbook). However, this method has the following problems and the like: (1) kinds and ratios of removable non-sugar components are limited; (2) non-sugar components remaining after the clarification process lower the efficiency of the crystallization of sugar (the remaining impurities inhibit crystal growth); (3) raw sugar and molasses are colored (colored substances are formed, when remaining soluble nitrogen components and sugar components are heated); (4) mineral components remaining in molasses in a large amount lower the fermentation performance of yeast during ethanol production; (5) anxiety over food safety is caused by the use of a flocculating agent, which is used to flocculate non-sugar components (suspended substances and some kinds of minerals) for flocculation and separation of the non-sugar components as porous large flocs; and (6) introduction of an ion-exchange resin is under consideration for removal, by adsorption, of non-sugar components (minerals, amino acids, pigments, and the like), but this increases the costs.

### SUMMARY OF INVENTION

An object of the present invention is to provide a method for producing sugar, capable of efficiently removing non-sugar components in a sugar juice, and lowering a color value of the sugar juice.

The present invention provides a method for producing sugar, comprising: adding ethanol to a sugar juice obtained by pressing a plant; removing precipitates formed by the ethanol addition; and crystallizing sugar from the sugar juice from which the precipitates are removed.

The method of the present invention makes it possible to efficiently remove non-sugar components in a sugar juice, and to lower a color value of the sugar juice.

### DESCRIPTION OF EMBODIMENT

A method for producing sugar of the present invention is characterized by comprising: adding ethanol to a sugar juice obtained by pressing a plant; removing precipitates formed by the ethanol addition; and crystallizing sugar from the sugar juice from which the precipitates are removed.

Examples of the plant include plants in which sugar components are accumulated, such as sugarcane, sugar beet, and sorghum. Sugarcane is preferable.

The pressing of the plant to obtain the sugar juice can be accomplished by methods known to those skilled in the art. Specifically, cane stem portions of reaped sugarcane are cut into pieces of 15 to 30 cm length with a cutter, and finely shredded with a shredder. The sugar juice is squeezed out with a roll mill. To increase the percentage of sugar components squeezed out, water is supplied to an end roll, and 95 to 97% of the sugar components are squeezed out. The obtained sugar juice mainly contains sucrose, glucose, fructose, and the like.

Note that the obtained sugar juice may be subjected to a heat treatment, or impurities may be flocculated and deposited by adding a chemical liquid of lime or the like. The heat treatment and the addition of lime are typical methods for removing non-sugar components with avoidance of degradation of sucrose and reducing sugars in the sugar juice as much as possible. As a result, non-sugar components (proteins, organic acids, suspended substances, some minerals) are made insoluble, and are then removed. Thus, a clarified juice can be obtained.

Subsequently, ethanol is added to the obtained sugar juice. As a result, non-sugar components and mineral components are precipitated. Hence, the non-sugar components and mineral components can be removed efficiently by removing these precipitates. In addition, the color value of the sugar juice can be lowered. Moreover, the color value of molasses can also be lowered, so that the color of the distillation waste liquid can also be reduced. The amount of ethanol added is preferably such that the liquid after the ethanol addition has an ethanol concentration of 40% by volume or more, and more preferably such that the liquid after the ethanol addition has an ethanol concentration of 50% by volume or more.

The precipitates produced by the ethanol addition are removed by an ordinary filter (a continuous rotary vacuum filter, a decanter, or the like).

Subsequently, sugar is crystallized from the sugar juice from which the precipitates are removed. The crystallization of sugar from the sugar juice can be accomplished by methods known to those skilled in the art. For example, the sugar juice is repeatedly heated and concentrated little by little (0.5 to 1 kl) under reduced pressure by suction, and sugar crystals having a certain size or larger are taken out. Then, the sugar crystals and the molasses are separated from each other with a centrifuge. Note that the sugar juice may be filtered and concentrated before the crystallization of the sugar. For example, the precipitates produced by heating and the addition of lime are filtered out with a continuous rotary vacuum filter, and a filtered juice is obtained. The filtered juice is mixed with a clarified juice, and heated and evaporated in a multiple-effect evaporator.

In the method for producing sugar of the present invention, ethanol may be separated from the sugar juice, before sugar is crystallized from the sugar juice. The separation of ethanol from the sugar juice can be accomplished by known methods to those skilled in the art. For example, ethanol is separated by distillation. When ethanol is separated by distillation, the sugar juice is concentrated simultaneously. Thus, it is no longer necessary to perform heating and concentrating in the sugar production. Hence, both of time and energy can be saved. In addition, the separated ethanol can be collected and reused.

In the method for producing sugar of the present invention, ethanol may be produced by fermenting the sugar juice with a microorganism, before sugar is crystallized from the sugar juice. The use of the obtained ethanol in the above-described ethanol addition step makes it possible to eliminate the need for purchase of ethanol, or to reduce the amount of purchase of ethanol.

The fermentation of the sugar juice can be accomplished by methods known to those skilled in the art. Examples thereof include a batch method in which a fermenting microorganism and a sugar juice are added at a predetermined ratio for fermentation; a continuous method in which a fermenting microorganism is immobilized, and then continuously supplied with a sugar juice for fermentation; and the like. Note that, in the method of the present invention, the mineral concentration is lowered by the above-described ethanol addition step. Hence, the fermentation can be carried out even with a salt-intolerant yeast without diluting the sugar juice.

The microorganism is preferably a microorganism having no sucrase. Examples of the microorganism having no sucrase include Saccharomyces dairenensis NBRC 0211, Saccharomyces transvaalensis NBRC 1625, Saccharomyces rosinii NBRC 10008, Zygosaccharomyces bisporus NBRC 1131, and the like. Meanwhile, among microorganisms having sucrase, it is possible to use a strain of a microorganism whose six sucrase genes (SUC1, SUC2, SUC3, SUC4, SUC6, and SUC7) are all or partially disrupted by gene manipulation.

In addition, the fermentation of the sugar juice may be conducted in the presence of a sucrase inhibitor. Examples of the sucrase inhibitor include silver ions, copper ions, mercury ions, lead ions, methyl-α-D-glucopyranoside, PCMB (p-chloromercuribenzoate), glucosyl-D-psicose, and the like.

In the method for producing sugar of the present invention, ethanol may be collected from the fermented sugar juice, before sugar is crystallized from the fermented sugar juice. The collection of ethanol from the fermented sugar juice can be accomplished by methods known to those skilled in the art. For example, the collecting is accomplished by separating ethanol by distillation. When ethanol is separated by distillation, the sugar juice is concentrated simultaneously. Thus, it is no longer necessary to perform heating and concentrating in the sugar production. Hence, both of time and energy can be saved.

### EXAMPLES

### Example 1

Test regarding Removal of Non-Sugar Components by Ethanol Addition

Approximately 1 ton of raw material stems of harvested sugarcane (KR98-1001 or S3-19) were shredded with a shredder, and then pressed with a three-roll mill. Thus, approximately 800L of a pressed juice was obtained.

To check the effect of removing non-sugar components, conventional heat.lime treatments and ethanol treatments were conducted. To an Erlenmeyer flask, 300 ml of the pressed juice was transferred, and boiled on a hot plate (Advantec, SR-550). Then, slaked lime Ca(OH)2 was add thereto to adjust the pH to 7.0, and impurities were flocculated (a heat.lime treatment). The pressed juice subjected to the heat.lime treatment was allowed to stand until room temperature was reached, and ethanol (special grade, 99.5%) was add thereto to be 30 to 50% by volume, followed by thorough stirring with a stirrer (ethanol treatment). Each pressed juice subjected to the heat.lime treatment and the ethanol treatment was centrifuged (1673 g, 5 minutes), and then the supernatant was filtered through a filter paper (Whatman, No. 54). The filtrate was concentrated with a rotary evaporator. Thus, a syrup having a Bx of about 60 was obtained.

To check the effects of the ethanol treatments, the purity, color value, turbidity, and mineral content of each of the obtained syrups were measured. The purity was measured by liquid chromatography. For the liquid chromatography, a column Sugar SC1011, Shodex, was used, and pure water was used as the mobile phase. The column oven temperature was set to 80° C. The color value and the turbidity were measured according to ICUMSA GS⅓-7 (ICUMSA, 2007a), and ICUMSA GS7-21 (ICUMSA, 2007b), respectively. The mineral content was measured after incineration at 600° C. for 30 minutes by use of a muffle furnace (EPTR-26K, Isuzu).

The following Table 1 shows results of the purities after the ethanol treatments. The effects were expressed in relative ratios to the standard, where the value of the conventional method is taken as 100. Treatments with 50% by volume of ethanol achieved purities of 75.3% and 69.4% for KR98-1001 and S3-19 were, respectively, which were 102.9% and 104.4% in terms of relative ratio to a standard, indicating that the purities were improved. However, when the ethanol concentration was lowered, a certain effect was observed in a treatment with 40% by volume of ethanol on KR98-1001, but the effects observed in the other treatments were merely at the same level of the conventional method.

TABLE 1

| | | | Relative | | Relative | | Relative |
|---|---|---|---|---|---|---|---|
| | Heat · Lime | E50 | ratio | E40 | ratio | E30 | ratio |
| KR98- | 73.1 | 75.3 | 102.9 | 74.8 | 102.3 | 74.3 | 101.6 |
| 1001 | | | | | | | |
| S3-19 | 66.5 | 69.4 | 104.4 | 66.8 | 100.5 | 65.9 | 99.1 |

E50, E40, and E30 show effects achieved when ethanol was added to be 50, 40, and 30% by volume, respectively. The relative ratio is a value where the value of the heat.lime treatment is taken as 100.

The following Table 2 shows results of the color values after the ethanol treatments.

TABLE 2

| | | | | | | | Rel- |
|---|---|---|---|---|---|---|---|
| | Heat · | | Relative | | Relative | | ative |
| | Lime | E50 | ratio | E40 | ratio | E30 | ratio |
| KR98- | 20787 | 16719 | 80.4 | 17582 | 84.6 | 17212 | 82.8 |
| 1001 | | | | | | | |
| S3-19 | 18479 | 16134 | 87.3 | 17465 | 94.5 | 20077 | 108.6 |

The following Table 3 shows results of the turbidities after the ethanol treatments. The treatments with 50% by volume of ethanol achieved turbidities of 2.3 and 2.2 for KR98-1001 and S3-19, respectively, which were 69.7% and 59.5% in terms of relative ratio to the standard. In addition, the treatments with 40% by volume of ethanol achieved turbidities of 2.5 and 2.6 for KR98-1001 and S3-19, respectively, which were 75.8% and 70.3 in terms of relative ratio to the standard. These turbidities were improved by these treatments. However, the effects observed in the treatments with 30% by volume of ethanol were merely at the same level of the conventional method.

TABLE 3

| | | | Relative | | Relative | | Relative |
|---|---|---|---|---|---|---|---|
| | Heat · Lime | E50 | ratio | E40 | ratio | E30 | ratio |
| KR98- | 3.3 | 2.3 | 69.7 | 2.5 | 75.8 | 3.1 | 93.9 |
| 1001 | | | | | | | |
| S3-19 | 3.7 | 2.2 | 59.5 | 2.6 | 70.3 | 4.1 | 110.8 |

The following Table 4 shows results of the mineral contents after the ethanol treatments. The treatments with 50% by volume of ethanol achieved mineral concentrations of 90.1 and 65.2 g/kg solid for KR98-1001 and S3-19, respectively, which were 82.1% and 74.1 in terms of relative ratio to the standard, indicating that the minerals were removed. However, when the ethanol concentration was lowered, a certain effect was observed in the treatment with 40% by volume of ethanol on S3-19, but the effects observed in the other treatments were merely at the same level of the conventional method.

TABLE 4
Mineral (g/kg solid)

| | | | Relative | | Relative | | Relative |
|---|---|---|---|---|---|---|---|
| | Heat · Lime | E50 | ratio | E40 | ratio | E30 | ratio |
| KR98- | 109.7 | 90.1 | 82.1 | 96.0 | 87.5 | 105.8 | 96.4 |
| 1001 | | | | | | | |
| S3-19 | 88.0 | 65.2 | 74.1 | 69.7 | 79.2 | 83.8 | 95.2 |

The above-described results show that a treatment with 40% by volume or more of ethanol achieves a certain effect, and a treatment with 50% by volume or more of ethanol is more effective in order to improve the purity, color value, turbidity, and mineral content.

1. A method for producing sugar, comprising the steps of:
adding ethanol to a sugar juice obtained by pressing a plant;
removing precipitates formed by the ethanol addition; and
crystallizing sugar from the sugar juice from which the precipitates are removed.

2. The method for producing sugar according to claim 1, wherein in the step of adding ethanol to the sugar juice obtained by pressing the plant, the ethanol is added such that a liquid after the ethanol addition has an ethanol concentration of 40% by volume or more.

3. The method for producing sugar according to claim 1, wherein ethanol is separated from the sugar juice before sugar is crystallized from the sugar juice.

4. The method for producing sugar according to claim 3, wherein the sugar juice is fermented with a microorganism before sugar is crystallized from the sugar juice.

5. The method for producing sugar according to claim 4, wherein the sugar juice is fermented with a microorganism having no sucrase.

6. The method for producing sugar according to claim 4, wherein the sugar juice is fermented in the presence of a sucrase inhibitor.

7. The method for producing sugar according to claim 4, wherein ethanol is collected from the fermented sugar juice before sugar is crystallized from the fermented sugar juice.

8. The method for producing sugar according to claim 2, wherein ethanol is separated from the sugar juice before sugar is crystallized from the sugar juice.

9. The method for producing sugar according to claim 8, wherein the sugar juice is fermented with a microorganism before sugar is crystallized from the sugar juice.

10. The method for producing sugar according to claim 9, wherein the sugar juice is fermented with a microorganism having no sucrase.

11. The method for producing sugar according to claim 9, wherein the sugar juice is fermented in the presence of a sucrase inhibitor.

12. The method for producing sugar according to claim 9 , wherein ethanol is collected from the fermented sugar juice before sugar is crystallized from the fermented sugar juice.
